(19) United States
(12) Patent Application Publication
(10) Pub. No.: US 2020/0397544 A1
(43) Pub. Date: Dec. 24, 2020

(54) METHOD FOR PREPARING PRODUCTION-SUITABLE 3D PRINTED PROSTHESIS USING POLYMERIZABLE MONOMERS

(71) Applicants: Ronald A. BULARD, Ardmore, OK (US); PARK DENTAL RESEARCH CORPORATION, Ardmore, OK (US)

(72) Inventor: Ronald A. BULARD, Ardmore, OK (US)

(73) Assignee: Park Dental Research Corporation, Ardmore, OK (US)

(21) Appl. No.: 16/643,304

(22) PCT Filed: Sep. 4, 2018
(86) PCT No.: PCT/US2018/049341

§ 371 Date: Feb. 28, 2020

Related U.S. Application Data
(60) Provisional application No. 62/553,151, filed on Sep. 1, 2017.

(51) Int. Cl. A61C 13/087; A61C 13/00; A61K 6/889; B33Y 10/00; B33Y 80/00; B29C 64/129

(52) U.S. Cl. CPC A61C 13/087; A61C 13/0019; A61C 13/0013; B29L 2031/7532; B33Y 10/00; B33Y 80/00; B29C 64/129; A61K 6/889

(57) ABSTRACT

A method of preparing a dental prosthesis comprises:
(a) 3D printing a dental prosthesis or a component of a dental prosthesis using a polymerizable resin ink with layer-by-layer curing with light; and thereafter
(b) subjecting the dental prosthesis or component thereof obtained in (a) to ionizing radiation radiation.

Also disclosed is a 3D printed dental prosthesis or component thereof prepared by the inventive method; and a method of providing a patient in need thereof with the 3D printed dental prosthesis or component thereof by inserting same into the oral cavity of the patient.

### BACKGROUND OF THE INVENTION

### 1. Field of the Invention

The present invention relates to a method for preparing a production-suitable 3D printed prosthesis from polymerizable monomers.

### 2. Description of Related Art

3D printing has been used to print dental prostheses, for example, dentures and implants. However, where the printing ink does not comprise a metal, for example, titanium, but, rather, comprises polymerizable resins, the dental prostheses are weak and often suffer the problem that in use they cannot withstand the forces of mastication.

For example, in stereolithography (SLA), a laser is used to solidify liquid resin with ultraviolet light. The laser beam draws out a slice of the developing 3D part to cure the liquid resin layer by layer, ultimately generating the 3D part.

Most resins, when they cure, are usually very brittle, and cannot withstand much force, so SLA printing is usually useful when it comes to prototyping, but not production. This is a significant limitation on the process, since the attractiveness of SLA printers is that their precision allows printing of very intricate, delicate structures, which makes the technique an ideal candidate for the 3D printing of dental prostheses were it not for the aforementioned limitations.

Accordingly, there remains a need to find ways of increasing the strength of dental prostheses 3D printed from polymerizable resin inks.

### SUMMARY OF THE INVENTION

These and other objects were met with the present invention, which relates in a first embodiment to a method of preparing a dental prosthesis comprising:
(a) 3D printing a dental prosthesis or a component of a dental prosthesis using a polymerizable resin ink with layer-by-layer curing with light; and thereafter
(b) subjecting the dental prosthesis or component thereof obtained in (a) to ionizing radiation.

The invention relates in a second embodiment to a dental prosthesis or component thereof prepared by the inventive process.

The invention relates in a third embodiment to a method of using the dental prosthesis or component thereof prepared by the inventive process to treat a patient in need of such treating.

### DETAILED DESCRIPTION OF THE INVENTION

Applicant has surprisingly and unexpectedly discovered that subjecting the light-cured 3D printed dental prosthesis or component thereof to a post-curing treatment with ionizing radiation, especially gamma radiation, most preferably Cobalt-60 radiation, which is commonly used for medical sterilization, can change the nature of the structure of the light-cured 3D printed dental prosthesis or component thereof, making the light-cured 3D printed dental prosthesis or component thereof much harder and stronger than it was following the initial printing despite the fact that the initial printing involved light-curing. The structural change is such that the initially printed construct, which lacks sufficient strength for production purposes, is rendered sufficiently strong that it can be used for production purposes, thereby overcoming the above-noted weakness of known non-metal 3D printed dental prosthetics or components thereof.

Suitable polymerizable resins are well-known in the art, as are polymerizable compositions containing them, the ingredients of such compositions, including suitable monomers and photoinitiators, and the operable conditions for preparing light-cured 3D printed dental prostheses and components thereof. Accordingly, these details are not repeated here. See, for example, the following patent publications: US 2009/0148813; US 2014/0131908; US 2014/0239527; US 2016/0113846; US 2016/0288376; and US 2016/0332367, the entire contents of which published applications are hereby incorporated herein by reference. The present invention is constructed to cover separately the use of all 3D printed dental prostheses and components thereof described therein, or any polymerizable compositions described therein, whether the 3D printed dental prostheses, components thereof, or polymerizable compositions are described generically or by working example, combined with the Cobalt-60 post-production treatment described herein. Preferably, the polymerizable resin ink comprises polymerizable (meth)acrylate monomers or any other resin capable of being converted from a liquid or powder to a solid using 3D printing.

Examples of the ionizing radiation to be applied to the 3D printed dental prosthesis or component thereof include alpha-rays, beta-rays, gamma-rays, electron beams, neutron rays, and X-rays. Among these, a gamma-ray of cobalt 60 or an electron beam is preferred, with cobalt-60 being the most preferred.

The irradiation of ionizing radiation is performed using an ionizing radiation irradiation apparatus, and the dose of irradiation is usually 5 to 300 kGy, preferably 20 to 60 kGy. The time is that necessary to bring about the aforementioned structural changes, which depends on the dose selected, is generally anywhere from a few seconds, for example, 10 seconds, or 30 seconds, or 60 seconds, until an hour or more, preferably from 40-50 minutes.

In a most preferred embodiment, the 3D printing is of a polymerizable mixture of (meth)acrylates and a photoinitiator built up layer-by-layer to form a dental prosthesis, especially a denture, under the curing effect of UV-light, and the dental prosthesis so produced is subjected to a dose of 20 to 60 kGy Cobalt-60 for 40-50 minutes.

An advantage of the use of the present invention, for example, Cobalt-60, is that the irradiators used produce very little heat, if any, and, therefore, the overall process involves little or no heat. This is different than, for instance, crosslinking with electromagnetic curing methods, for example, microwaves, which generate significant heat to cure the resins.

The invention will now be described in greater detail with reference to the following non-limited example.

### Example

A polymerizable dental material is prepared by combining polyester acrylate, aliphatic epoxy diacrylate, trimethylolpropanetriacrylate, iso-bornyl acrylate, and phosphine oxide, methanone. Two dentures are 3D printed from the polymerizable dental material using the Juell™ 3D-2 printer available from Park Dental Research Corporation, Ardmore, Okla., UV-light-curing layer-by-layer during the build. Upon completion the first denture is set aside while the second denture is subjected to a 25 kGy dose of Cobalt-60 radiation for a period of about 45 minutes.

At the end of the time, the two dentures are subjected to the average forces occurring during mastication. Whereas the first denture breaks, the second denture remains unaffected, thereby, indicating the second, Cobalt-60-treated denture is significantly stronger than the first denture.

While the present invention has been described in conjunction with the specific embodiments set forth above, many alternatives, modifications and other variations thereof will be apparent to those of ordinary skill in the art. All such alternatives, modifications and variations are intended to fall within the spirit and scope of the present invention.

1. A method of preparing a dental prosthesis comprising:
(a) 3D printing a dental prosthesis or a component of a dental prosthesis using a polymerizable resin ink with layer-by-layer curing with light; and thereafter
(b) subjecting the dental prosthesis or component thereof obtained in (a) to ionizing radiation radiation.

2. The method according to claim 1, wherein the polymerizable resin ink comprises polymerizable (meth)acrylate monomers.

3. The method according claim 1, wherein the curing with light involves ultraviolet light.

4. The method according to claim 1, wherein the ionizing radiation is gamma radiation.

5. The method according to claim 4, wherein the gamma radiation is Cobalt-60.

6. The method according to claim 5, wherein the Cobalt-60 is used in a dosage of 20-60 kGy.

7. The method according to claim 6, wherein the dosage is about 25 kGy.

8. A dental prosthesis prepared by a method according to claim 1.

9. A method of providing a dental patient with a dental prosthesis, the method comprising:
(a) preparing a dental prosthesis according to claim 1; and
(b) installing the dental prosthesis into the oral cavity of said patient.
